(19) United States
(12) Patent Application Publication
(10) Pub. No.: US 2025/0313796 A1
(43) Pub. Date: Oct. 9, 2025

(54) APPARATUS AND METHOD FOR ISOLATING INDIVIDUAL COLONIES THROUGH AUTOMATIC MULTI-CHANNEL STREAKING IN HIGH-PRESSURE ENVIRONMENT

(71) Applicant: Guangdong University of Technology, Guangzhou (CN)

(72) Inventors: Jingchun FENG, Guangzhou (CN); Si ZHANG, Guangzhou (CN); Song ZHONG, Guangzhou (CN); Yi WANG, Guangzhou (CN); Zhifeng YANG, Guangzhou (CN)

(73) Assignee: Guangdong University of Technology, Guangzhou (CN)

(21) Appl. No.: 18/625,237

(22) Filed: Apr. 3, 2024

(51) Int. Cl. C12N 1/02; C12M 1/00; C12M 1/26; C12M 1/34; C12M 1/36; C12N 1/20

(52) U.S. Cl. CPC C12N 1/02; C12M 29/14; C12M 33/04; C12M 41/12; C12M 41/40; C12M 41/48; C12N 1/20

(57) ABSTRACT

An apparatus and method for isolating individual marine microorganism colonies through automatic multi-channel streaking in a high-pressure environment is provided. The apparatus includes an enrichment system, an isolation operation incubator, a central control system, a temperature control unit and a pressure control unit. The high-pressure pure culture apparatus and method increases microorganism survival activity and preserves their phenotype from changes caused by atmospheric pressure isolation and culture.

### TECHNICAL FIELD

The present invention relates to the technical field of marine microorganism isolation, and in particular to an apparatus and method for isolating individual colonies through automatic multi-channel streaking in a high-pressure environment.

### BACKGROUND

The ocean is the largest single ecosystem on earth. Marine sediments cover more than 70% of the earth and contain as much biomass as seawater. In recent decades, with the development and advancement of scientific research technology and deep-sea submarines, scientists have had the opportunity to obtain samples of major deep-sea seabed sediments to better understand the classification and abundance of microorganisms in seabed sediments. According to research, it is estimated that the bacteria in marine sediments currently account for 0.23-3.6% of the bacteria on the earth. It is estimated that seafloor microorganisms account for five-sixths of the earth's total biomass and one-third of its living biomass. These microbial communities process both organic and inorganic carbon and help cycle nutrients such as sulfur, nitrogen, sulphur, and iron. Some of the more important core taxa play important roles in global biogeochemical cycles.

Based on 16S rRNA gene amplicon sequencing analysis, many uncultured taxa were found. One of the distinctive features of these uncultured taxa is that they dominate the deep sea. Given the importance of these uncultivated taxa to the entire ocean, there is a need to better understand and appreciate the diversity and ecological roles of these uncultivated taxa.

For the isolation of microorganisms in special marine habitats, existing technology is mainly carried out in normal pressure environments, and rarely isolates and cultivates individual colonies in high-pressure environments. Even in high-pressure environments, mechanical streaking is generally adopted or artificial streaking is performed to isolate individual colonies after pressure relief. Moreover, the type of culture medium used for each streaking is relatively single, resulting in low isolation efficiency and cumbersome operation process. In addition, the number of cultured microorganisms is less than 1% of that in the deep-sea environment. Certain difficulties in correctly understanding and utilizing marine resources still exist.

### SUMMARY

The objective of the present invention is as follows: in order to solve the problems existing in the prior art, the present invention provides an apparatus and method for isolating individual colonies through automatic multi-channel streaking in a high-pressure environment.

In order to solve the problems existing in the prior art, the present invention adopts the following technical solutions:

An apparatus for isolating individual colonies through automatic multi-channel streaking in a high-pressure environment, including an enrichment system, an isolation operation incubator, a central control system, a temperature control unit and a pressure control unit, wherein
the enrichment system is communicated with the isolation operation incubator, and the enrichment system, the isolation operation incubator, the temperature control unit and the pressure control unit are all electrically connected to the central control system;
the enrichment system is configured to cultivate microorganisms, and the enrichment system includes an enrichment kettle and a removable upper cover; and the enrichment system includes a gas inlet channel or a liquid inlet channel through which gas or liquid is correspondingly injected into the enrichment kettle to pressurize the enrichment kettle;
the central control system is configured to monitor environmental data changes in the high-pressure environment, and perform monitoring, real-time collection, processing, storage and image output; the temperature control unit is configured to detect and adjust temperature changes in the enrichment system and the isolation operation incubator; and the pressure control unit is configured to detect and adjust pressure changes in the enrichment system and the isolation operation incubator, and inject gas or liquid into the isolation operation incubator for pressurization;
the isolation operation incubator includes a culture chamber in which a plurality of culture dishes are arranged, the plurality of culture dishes are arranged vertically or staggered horizontally and vertically, and each of the culture dishes forms a channel.

As an improvement of the technical solution of the apparatus for isolating individual colonies through automatic multi-channel streaking in a high-pressure environment according to the present invention, the plurality of culture dishes are arranged in upper and lower layers, and the culture dishes in each layer are staggered;
a fixing rod perpendicular to a bottom of the culture chamber is provided in the culture chamber, and a plurality of cross bars are disposed transversely on the fixing rod;
each of the culture dishes is in a shape of a rectangular parallelepiped, fixing holes are provided on either side of the culture dish, a slope is provided on the other side of the culture dish, an upper edge of the slope forms an overflow line, baffles are provided on either side of the slope, eyelets are provided below the culture dish, the eyelets are provided at an end of the culture dish close to the slope, and the plurality of culture dishes form a complete channel in a vertical direction; and
the fixing hole is provided in such a way that it extends through the cross bar, a connecting rod is provided in the eyelet, and the connecting rod is configured to connect the culture dishes arranged in the upper and lower layers.

As an improvement of the technical solution of the apparatus for isolating individual colonies through automatic multi-channel streaking in a high-pressure environment according to the present invention, a vertical distance between each of the culture dishes is greater than or equal to a height of the culture dish itself; and when the culture dish is tilted, the culture dish forms a “Z” shape, and an inclined end of the culture dish in the upper layer is in internal contact with a non-inclined end of the culture dish in the lower layer.

As an improvement of the technical solution of the apparatus for isolating individual colonies through automatic multi-channel streaking in a high-pressure environment according to the present invention, at least three layers of culture dishes are provided in the culture chamber, and the at least three layers of culture dishes include a first isolation layer, a second isolation layer and a third isolation layer; and each of the isolation layers includes at least one of the culture dishes.

As an improvement of the technical solution of the apparatus for isolating individual colonies through automatic multi-channel streaking in a high-pressure environment according to the present invention, the isolation operation incubator is further connected to a dilution bottle, and sterile water is injected from the dilution bottle into the culture dish.

As an improvement of the technical solution of the apparatus for isolating individual colonies through automatic multi-channel streaking in a high-pressure environment according to the present invention, the enrichment system is communicated with the isolation operation incubator through a liquid supply pipe, and a micro-injection pump is further disposed to the liquid supply pipe; the enrichment system injects culture fluid into the isolation operation incubator through the liquid supply pipe, the micro-injection pump and spouts; and a number of the spouts is consistent with a number of culture dishes in a same plane; and
the culture chamber is further provided with a liquid accumulation tank, the liquid accumulation tank is in operative contact with the slope of the culture dish at the bottom layer; and the liquid accumulation tank is further communicated with a collection apparatus through a second pipe, and the second pipe is further provided with a valve.

As an improvement of the technical solution of the apparatus for isolating individual colonies through automatic multi-channel streaking in a high-pressure environment according to the present invention, the enrichment kettle includes a removable upper cover; a stirring rod is provided at the top inside the enrichment kettle; the gas inlet pipe and the liquid inlet pipe are equipped with valves; the enrichment kettle is further provided with a sampling port for a regulating valve; the enrichment kettle is placed in high-temperature or low-temperature water bath, or placed in an air heat exchange constant temperature room; and the enrichment system further includes a micro-injection pump and a liquid outlet pipe.

A method for isolating individual colonies through automatic multi-channel streaking in a high-pressure environment uses the above-mentioned apparatus for isolating individual colonies through automatic multi-channel streaking in a high-pressure environment.

The method includes the following steps:
S1. after concentration of a target bacterial solution in an enrichment process reaches a required level, entering into an isolation and culture process;
S2. sterilizing the isolation operation incubator to maintain sterility;
S3. filling a bottom of an isolation area with sterilized solid culture medium and placing a culture dish;
S4. turning on a water bath system to ensure that temperature of the isolation operation incubator is consistent with temperature conditions of microorganisms in a marine environment;
S5. injecting gas or liquid into the isolation operation incubator to pressurize the isolation operation incubator so that pressure conditions in the isolation operation incubator are consistent with pressure conditions of microorganisms living in the marine environment;
S6. injecting a microbial enrichment liquid from the enrichment kettle into the isolation operation incubator through the micro-injection pump, wherein bacterial liquid dripping on the first isolation layer automatically flows downward under gravity and passes through the second isolation layer and the third isolation layer in sequence and initially isolated into individual colonies during the flow process;
S7. injecting sterile water in the dilution bottle onto a flat plate of the first isolation layer through the micro-injection pump, wherein the sterile water flows downward under the action of gravity, and the sterile water dilutes the bacterial liquid originally flowing on the surface of the culture medium during the flow process and dilutes the microbial enrichment liquid to the maximum extent; and
S8. performing multi-channel isolation of individual colonies in sequence according to the steps of S6 and S7.

Beneficial effects of the present invention are set forth as follows.

1. In the present invention, an apparatus and method for enriching microorganisms and using automatic multi-channel streaking to isolate multiple types of individual colonies under environmental conditions such as in-situ temperature and pressure of the ocean are realized. That is, the present invention solves the problem of poor survival activity of a large number of microorganisms caused by the existing indoor pure culture technology methods being departed from the high pressure and extreme temperature environmental conditions in which microorganisms survive, and solves the problems of low isolation efficiency and complicated operations in high pressure environments.

2. Compared with the existing high-pressure isolation and culture technology, the present invention can realize isolation and culture of individual colonies through automatic multi-stage and area-dividing streaking and combine different types of culture medium to achieve the greatest degree of isolation of individual colonies and solve the problem of low isolation efficiency and complicated operations in high-pressure environments.

3. Compared with the existing isolation and culture technology, the present invention can effectively reduce the investment of professionals, and can carry out large-scale enrichment and isolated culture, improve the screening efficiency of difficult-to-cultivate microorganisms, and improve the screening and cultivation efficiency of functional bacteria in high-pressure environments.

4. The present invention does not require professional operators and can be applied in multiple cultivation scenarios such as research laboratories and scientific research ships, which has wide adaptability. The present invention does not require manual enrichment and streaking isolation operations by professionals and can carry out large-scale enrichment and sorting, reducing labor costs, realizing automated isolation and culture of microorganisms in high-pressure environments under in-situ pressure and temperature environmental conditions, and providing an important technical means for pure cultivation of microorganisms in high-pressure environments under in-situ conditions.

### BRIEF DESCRIPTION OF THE DRAWINGS

FIG. 1 is a schematic structural diagram of a first state of an apparatus for isolating individual colonies through automatic multi-channel streaking in a high-pressure environment according to the present invention;

FIG. 2 is a schematic structural diagram of a second state of the apparatus for isolating individual colonies through automatic multi-channel streaking in a high-pressure environment according to the present invention;

FIG. 3 is a top view of a multi-channel structure of the apparatus for isolating individual colonies through automatic multi-channel streaking in a high-pressure environment according to the present invention;

FIG. 4 is a front view of the multi-channel structure in the apparatus for isolating individual colonies through automatic multi-channel streaking in a high-pressure environment according to the present invention;

FIG. 5 is a schematic structural diagram of a culture dish in the apparatus for isolating individual colonies through automatic multi-channel streaking in a high-pressure environment according to the present invention;

FIG. 6 is a schematic diagram of a circuit module connection of a central control system in the apparatus for isolating individual colonies through automatic multi-channel streaking in a high-pressure environment according to the present invention; and

FIG. 7 is a schematic flow chart of a method for isolating individual colonies through automatic multi-channel streaking in a high-pressure environment according to the present invention.

Reference numerals: 1—Central control system; 2—Isolation operation incubator; 21—Upper cover; 211—Fixing rod; 212—Cross bar; 22—Culture chamber; 221—Culture dish; 222—Overflow line; 223—Slope—224—Baffle; 225—Fixing hole; 226—Eyelet; 227—Connecting rod; 23—Liquid accumulation tank; 24—Valve; 25—Liquid outlet; 26—Collection apparatus; 3—Temperature control unit; 31—Temperature sensor; 4—Pressure control unit; 41—Air compressor; 42—Booster pump; 43—Gas storage tank; 44—Pressure regulating valve; 45—gas inlet valve; 46—gas supply pipe; 47—Pressure sensor; 5—Enrichment system; 51—Enrichment kettle; 511—Removable upper cover; 52—Micro-injection pump 53—Liquid outlet pipe; 54—Spout; 6—Dilution bottle.

### DETAILED DESCRIPTION OF THE EMBODIMENTS

In order to make the objects, technical solutions and beneficial effects of the present invention clearer, the technical solutions in the embodiments of the present invention will be clearly and completely described below in conjunction with the accompanying drawings in the embodiments of the present invention. Obviously, the described embodiments are only some embodiments of the present invention, but not all embodiments.

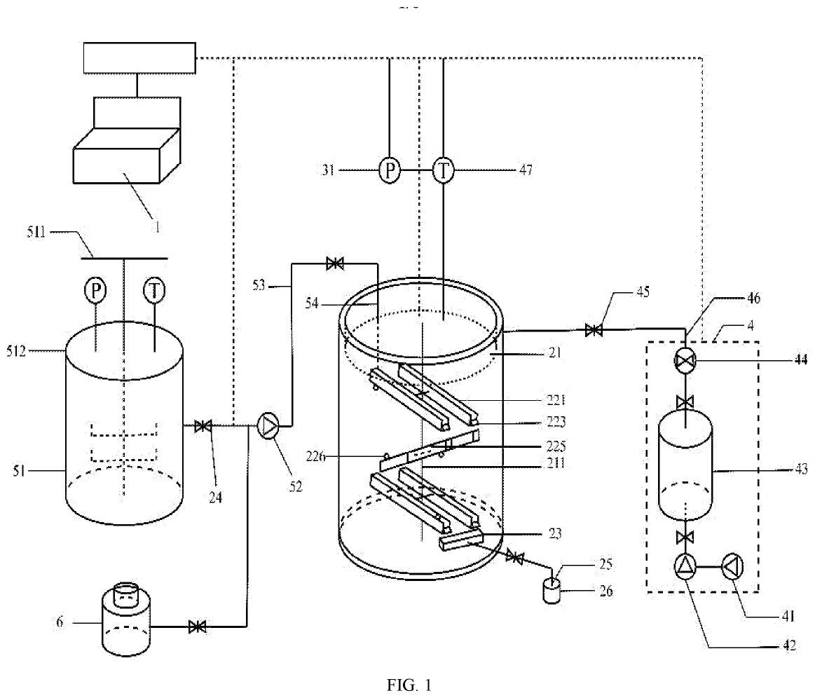

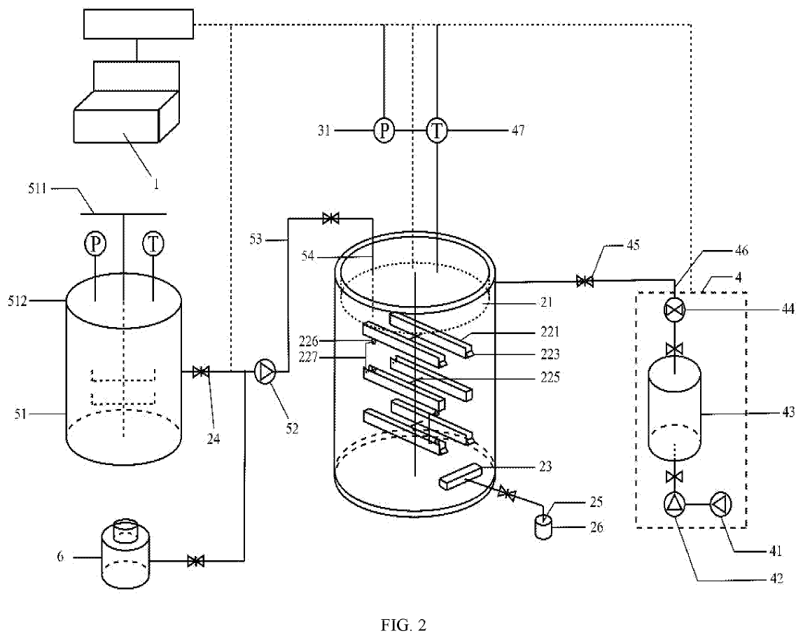

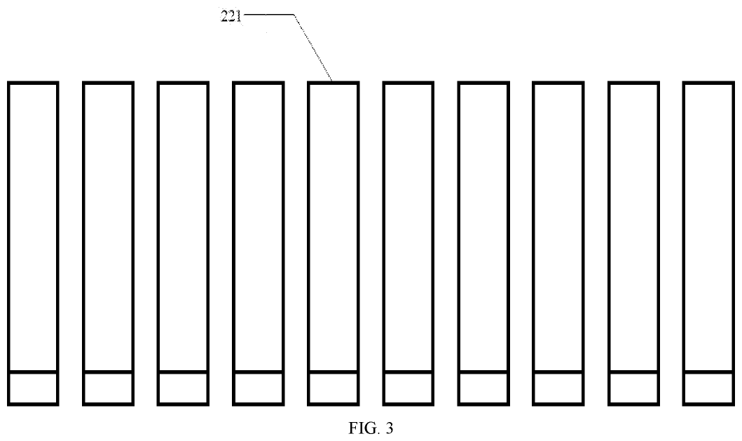

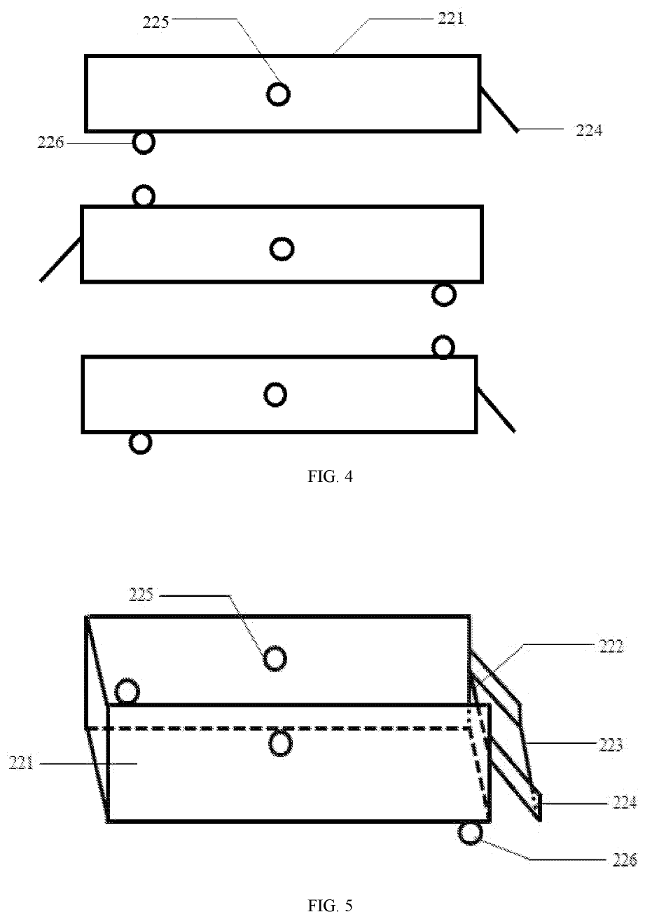

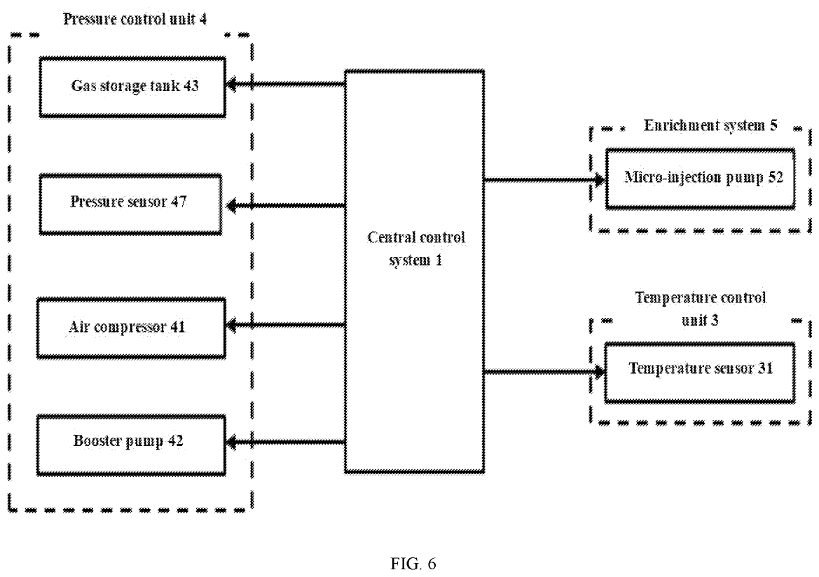

As shown in FIG. 1 to FIG. 6, an apparatus for isolating individual colonies through automatic multi-channel streaking in a high-pressure environment is characterized by including an enrichment system 5, an isolation operation incubator 2, a central control system 1, a temperature control unit 3, and a pressure control unit 4.

The enrichment system 5 is communicated with the isolation operation incubator 2, and the enrichment system 5, an isolation operation incubator 2, the temperature control unit 3, and the pressure control unit 4 are all electrically connected to the central control system 1.

The enrichment system 5 is configured to culture microorganisms, and the enrichment system 5 includes an enrichment kettle 51 and a removable upper cover 511; and the enrichment system 5 includes a gas inlet channel or a liquid inlet channel through which gas or liquid is correspondingly injected into the enrichment kettle 51 to pressurize the enrichment kettle 51.

The central control system 1 is configured to monitor changes in the environmental data in the high-pressure environment, and perform monitoring, real-time collection, processing, storage and image output; the temperature control unit 3 is configured to detect and adjust changes in the temperature in the enrichment system 5 and the isolation operation incubator 2; and the pressure control unit 4 is configured to detect and adjust changes in the pressure in the enrichment system 5 and the isolation operation incubator 2, and inject gas or liquid into the isolation operation incubator 2 for pressurization.

The isolation operation incubator 2 includes the incubation chamber 22, in which a plurality of culture dishes 211 are arranged, and the plurality of culture dishes 221 are arranged vertically or staggered horizontally and vertically, with each culture dish forming a channel.

As a first implementation of the present invention, the plurality of culture dishes 221 are arranged in upper and lower layers, and the culture dishes 221 at each layer are staggered; and a fixing rod 211 perpendicular to the bottom of the incubation chamber 22 is provided in the incubation chamber 22, and a plurality of cross bars 212 are disposed transversely on the fixing rod 211.

Each of the culture dishes 221 is in a shape of a rectangular parallelepiped; fixing holes 225 are provided on either side of the culture dish 221; a slope 223 is provided on the other side of the culture dish 221; an overflow line 222 is formed on an upper edge of the slope 223; baffles 224 are provided on either side of the slope 223; an eyelet 226 is provided on a lower side of the culture dish 221 and on an end of the culture dish 221 close to the slope 223; and the plurality of culture dishes 221 form a complete channel in a vertical direction. The fixing hole 225 is provided in such a way that it penetrates through the cross bars 212, and connecting rods 227 penetrate through the eyelets 226, and are configured to connect the culture dishes 221 arranged vertically in layers.

As a second implementation of the present invention, the vertical distance between every two culture dishes 221 is greater than or equal to the height of each culture dish 221 itself; and when the culture dishes 221 are tilted, the culture dishes 221 form a “Z” shape, and an inclined end of each culture dish 221 in the upper layer is in internal contact with an end of the corresponding culture dish 221 without the slope 223 in the lower layer.

As a third implementation of the present invention, at least three layers of culture dishes 221 are provided in the incubation chamber 22, and the at least three layers of culture dishes 221 include a first isolation layer, a second isolation layer, and a third isolation layer; and each of the isolation layers includes at least one culture dish 221.

As a fourth implementation of the present invention, the isolation operation incubator 2 is further connected to a dilution bottle 6, by means of which sterile water is injected into the culture dishes 221.

As a fifth implementation of the present invention, the enrichment system 5 is communicated with the isolation operation incubator 2 through a liquid supply pipe, and a micro-injection pump 52 is further disposed to the liquid supply pipe; the enrichment system 5 injects culture fluid into the isolation operation incubator 2 by means of the liquid supply pipe, the micro-injection pump 52, and spouts 54; and the number of the spouts 54 is the same as the number of the culture dishes 221 in a same plane.

The incubation chamber 22 is further provided therein with a liquid accumulation tank 23, which is kept in operative contact with the slope 223 of the culture dish 221 at the bottommost layer; and the liquid accumulation tank 223 is also communicated with a collection apparatus 26 through a second pipe, on which a valve 24 is further provided.

As a sixth implementation of the present invention, the enrichment kettle 51 includes a removable upper cover 511; a stirring rod is provided at the top inside the enrichment kettle 51; the gas inlet pipe and the liquid inlet pipe are both equipped with valves 24; the enrichment kettle 51 is further provided with a sampling port for a regulating valve; the enrichment kettle 51 is placed in a high-temperature or low-temperature water bath, or placed in an air heat exchange constant temperature room; and the enrichment system 5 further includes a micro-injection pump 52 and a liquid outlet pipe 53.

In the apparatus for isolating individual colonies through automatic multi-channel streaking in the high-pressure environment of the present invention, target microorganisms with high purity are cultured by the enrichment system 5, and then, individual microorganism colonies are isolated by performing automatic multi-stage streaking on a microbial liquid on different solid culture media. The entire process of enrichment and isolation is carried out under the original pressure and temperature environmental conditions of the microorganisms.

The pressure control unit 4 is mainly configured to inject a gas into the microorganism isolation incubation chamber 22 for pressurization, such that the pressure environment in the isolation operation incubator 2 is the same as the original pressure value of the microorganisms in the sea, and at the same time, changes in the pressure within the isolation operation incubator 2 are monitored. The pressure control unit 4 includes a pressure sensor 47 and a pressurization system; changes in the pressure within the incubation chamber 22 are monitored in real time by the pressure sensor 47; and the incubation chamber 22 is pressurized and depressurized by means of active gas injection/discharge, to keep the pressure values in the incubator chamber 22 and in an isolation chamber the same as the marine environment conditions where these microorganisms grow.

The pressurization system mainly includes an air compressor 41, a booster pump 42, a gas storage tank 43, a pressure regulating valve 44, a gas inlet valve 45, a gas supply pipe 46, and accessories such as pipes and valves. A temperature control system involved in the present invention is mainly to monitor changes in the temperature within the enrichment tank 51 and the isolation operation incubator 2. The central control system 1 involved in the present invention includes a data collector, a central data processing unit, an operating computer or the like to allow for monitoring, real-time collection, processing, storage, image output or other functions in relation to changes in various environmental data information during the enrichment, isolation, and purification of the microorganisms enriched in the high-pressure environment.

Specifically, in the present invention, target microorganisms are cultured by means of the enrichment system 5 to obtain target microbial flora with high purity, which are subsequently fed into the isolation operation incubator 2 with the pressure held for solid culture isolation, and the microbial flora are simultaneously screened by means of combined processes using different culture media and environmental conditions to obtain pure cultured microbial strains.

The enrichment system 5 includes an enrichment kettle 51, which is structurally designed with a removable upper cover 511 to facilitate placement of culture substrates and sterilizing operations. The top of the enrichment tank 51 is designed with a stirring rod, which may enhance mass transfer through manual or mechanical stirring to enhance the reaction process of the substrates during the culture process and increase the energy and nutrient utilization efficiency of microorganisms. The body of the enrichment kettle 51 is equipped with pressure and temperature sensors 31 to monitor the change in the temperature and pressure within the enrichment kettle 51 in real time. The constant temperature condition of the enrichment kettle 51 is mainly maintained by placing the enrichment kettle 51 in a temperature-monitored high/low-temperature water bath, whereby the enrichment kettle 51 is maintained at a constant temperature by means of its heat exchange with a water bath system. Alternatively, the enrichment kettle 51 is placed in an air heat exchange type constant temperature room, and is pressurized by injecting gas (or inert gas) or liquid necessary for culture into the closed enrichment kettle 51 by means of the gas and liquid inlet channels (each provided with a regulating valve) arranged on the top of the enrichment kettle 51, to thereby keep the pressure value in the enrichment vessel 51 the same as that of the actual original high-pressure environment. The enrichment kettle 51 is provided with a sampling port for the regulating valve, and the sampling port is configured for analysis and detection of samples taken during the enrichment process, so as to adjust the corresponding environmental parameters and optimize the process of enrichment culture.

Further, the enrichment system 5 includes an enrichment kettle 51, a liquid supply pipe, a micro-injection pump 52, valves 24, a liquid outlet pipe 53, and spouts 54. A microbial liquid that needs to be isolated in the isolation operation incubator 2 is supplied from the enrichment kettle 51 to the spouts 54 through the liquid supply pipe and the micro-injection pump 52. The number of the spouts 54 is the same as that of the plurality of channels, and the spouts 54 are disposed directly above the leftmost of the plurality of channels for the purpose of allowing each channel to obtain the same amount of microbial liquid. In order to ensure the pressure stability of the whole apparatus, the pipes are each provided with the valve 24. The isolation operation incubator 2 includes an upper cover, a lower isolation region, and bottom feet. The isolation region is arranged in the incubation chamber 22, and the cover and the isolation region are connected by buckles.

Highly purified target microorganisms are obtained by long-term indoor enrichment culture in an early stage and under the stress of directional nutrient supply. For the isolation and transfer of the target microorganisms, an enrichment liquid in the enrichment tank 51 may be taken out via the sampling port and pumped into the isolation operation incubator 2 by means of the micro-injection pump 52. During the entire process of enrichment, isolation and purification, the environment conditions of temperature and pressure in the incubator are the same as the environmental conditions of microorganisms in the deep sea, thereby ensuring the effectiveness of enrichment culture.

The isolation operation incubator 2 works based on the principle of isolating the microbial liquid on the solid culture medium through automatic multi-channel streaking, where the microbial enrichment culture liquid in the enrichment system 5 is injected into the uppermost layer of culture dishes 221 in the isolation operation incubator 2 by means of the micro-injection pump 52; individual colonies are initially isolated under the gravity of the microbial liquid itself; then with a dilution bottle 6, sterile water is injected into the culture dishes 221, such that non-isolated individual colonies on the culture dishes 221 are washed up and isolated to gradually form individual colonies on the culture dishes 221.

The present invention further includes an isolation holder. For the sake of effective space utilization, the present invention uses a truncated cone-like or cylindrical microorganism isolation holder, which maximizes the area for microorganism isolation and culture in a limited space. The isolation region is provided therein with a culture dish 221; the eyelet 226 of the culture dish 221 is connected to the cross bar 212 on the fixing rod 211 by fastening means, such that the connected culture dish 221 can freely move left and right on the cross bar 212. The culture dish 221 is not symmetrically positioned on the cross bar 212, where one end of the culture dish 221 provided with the slope 223 is closer to the cross bar 212 than the other end of the culture dish, which can ensure that the culture dish 221 tilts in a horizontal direction under gravity. The number of the culture dish 221 is greater than or equal to one depending on the experimental requirements. In order to ensure good fluidity of the microbial liquid in the culture dish, the vertical distance between every two culture dishes 221 is greater than or equal to the height of each culture dish 221 itself; and the culture dishes 221 on each layer are staggered to ensure that the culture dishes 221 may form a “Z” shape under the gravity, and the tilted end of each culture dish 221 on the upper layer is in internal contact with the end of the corresponding culture dish without the slope 223 on the lower layer.

The incubation chamber 22 is composed of the culture dish 221, the overflow line 222, the slope 223, the baffle 224, the fixing hole 225, eyelets 226, and the connecting rod 227. The eyelet 226 is circular for the purpose of matching the cross bar 212 on the fixing rod 211, such that the culture dish 221 can be disposed on the fixing rod 211; the overflow line 222 is lower than the height of the culture dish 221 for the purpose of allowing the superfluous culture medium to enter a next culture medium across the overflow line 222 during the pouring of the culture medium; the baffle 224 is disposed on the slope 223 mainly to prevent the culture medium from flowing out while flowing into a next culture dish 221 via the slope 223; the eyelets 226 are disposed on the side surface of the culture dish 221, each on either of the upper and lower sides, to match the connecting rod 227 for the purpose of keeping the culture dish 221 in the horizontal direction during pouring of the culture medium, such that the culture medium in the culture dish 221 may be kept horizontal and the superfluous culture medium may flow into the next culture dish 221 via the slope 223, allowing the superfluous culture medium to enter a liquid accumulation tank 23 finally. The isolation operation incubator 2 further includes the liquid accumulation tank 23, valves 24, a liquid outlet 25, and a collection apparatus 26. The liquid accumulation tank 23 is mainly configured to collect the superfluous culture medium and microbial liquid, and is kept in operative contact with the slope 223 of the culture dish 221 on the bottommost multi-channel layer, such that the superfluous culture medium and microbial liquid may flow into the liquid accumulation tank 23; and the double valves 24 of the liquid outlet 25 may be controlled to allow the superfluous microbial liquid to flow to the collection apparatus 26 via the liquid outlet 25.

The culture dish 221 involved in the present invention is in a multi-channel arrangement, with each channel disposed vertically and the plurality of channels disposed horizontally; each incubation chamber 22 is fixed by connection to the fixing rod 211 for the purpose of arranging a plurality of isolation incubation channels in the isolation operation incubator to improve the isolation efficiency for individual microorganisms; meanwhile, each channel may be filled with a solid culture medium containing culture substrates of different nutrient ratios. The number of isolation channels should be the same as the number of liquid inlets.

The technology employed in the present invention is to isolate various types of individual colonies through automatic multi-stage streaking in a high-pressure environment. First, the enrichment kettle 51 and its attached pipes and valves are sterilized; then, substrates to be cultured, such as deep-sea sediments, macro-biological tissues symbiotic with microorganisms and their extracts, are sequentially fed; then, a nutrient solution necessary for incubation is fed from a liquid injection port; and a gas necessary for the incubation (or an inert gas, if not needed) is injected from a gas injection port to increase the value of pressure within the incubation chamber to be the same as that in the actual environmental conditions of the deep sea. During the culture process, stirring is performed by the stirring apparatus at the top to enhance the mass transfer effect and optimize the culture process.

As an embodiment of this implementation, at least three layers of culture dishes 221 are provided in the isolation operation incubator 2, including a first isolation layer, a second isolation layer, and a third isolation layer, respectively. The number of the isolation layers can be determined according to the experimental requirement, which is not limited here.

After it is identified that the concentration of a target microbial liquid in an enrichment process reaches a required level, an isolation and culture process is initiated. The process of isolation and culture is mainly as follows. First, the isolation operation incubator 2 and all internal devices therein as well as related pipes and valves are sterilized to maintain sterility. The culture dishes 221 are positioned on the fixing rod 211, and the connecting rods 227 are used to connect the eyelets 226 on the sides of the culture dishes 221, such that the culture dishes 221 are at a horizontal position; then, a sterilized solid culture medium is slowly filled to the culture dish 221 on the upper layer in the incubation chamber 22; and the superfluous culture medium in the first isolation layer flows into the next layer, and so on, until each culture dish 221 is filled with an equal amount of medium. The superfluous medium during the pouring flows into the liquid accumulation tank 23. After the culture medium is solidified, the connecting means is removed, such that the culture dishes 221 are tilted under gravity, and so on, and finally the respective channels form a “Z” shape. Then, the upper cover 21 is closed; the high/low-temperature water bath system is turned on to ensure that temperature of the isolation operation incubator 2 is the same as temperature conditions of microorganisms in a marine environment. Then, gas or liquid is injected via the gas injection port for pressurization to allow the pressure conditions in the isolation operation incubator 2 to be the same as the pressure conditions of microorganisms living in the marine environment. After ensuring that all system components are working normally, the micro-injection pump 52 is turned on to inject the microbial enrichment liquid from the enrichment kettle 51 into the isolation operation incubator 2, and the microbial enrichment solution drips on the surface of the culture medium on the left of the first isolation layer.

The individual colonies in the microbial enrichment liquid are isolated three times. The first isolation is performed in the first isolation layer, where the microbial liquid dripping to the first isolation layer automatically flows downward under gravity and passes through the second isolation layer and the third isolation layer in sequence, and the microbial liquid is initially isolated into individual colonies during the flow process. After a period of time (which is adjusted as appropriate), sterile water in the dilution bottle 6 is slowly injected by the micro-injection pump 52 onto a flat plate of the first isolation layer in the isolation operation incubator; the sterile water gradually flows downward under gravity (the volume of sterile water injected may be set as appropriate depending on the number of colonies in the enrichment liquid); and the sterile water dilutes the microbial liquid originally flowing on the surface of the culture medium during the flow process, such that the microbial enrichment liquid is diluted to the maximum extent. In order to prevent the microbial liquid on the surface of the culture medium from being completely washed off, the sterile water injected should not be excessive, and the volume of the sterile water injected should be less than the volume of the sterile water flowing out to the liquid accumulation tank 23. Each channel is operated according to the above-mentioned method, such that the automatic multi-stage isolation of individual colonies under the high-pressure environment is completed step by step under gravity. Each channel gradually dilutes the microbial liquid on the surface of the culture medium, such that the probability of isolating the microbial enrichment liquid into individual colonies is improved by multiple isolation, instead of only one isolation, and depending on type of the culture medium in each channel, many types of individual colonies can be obtained.

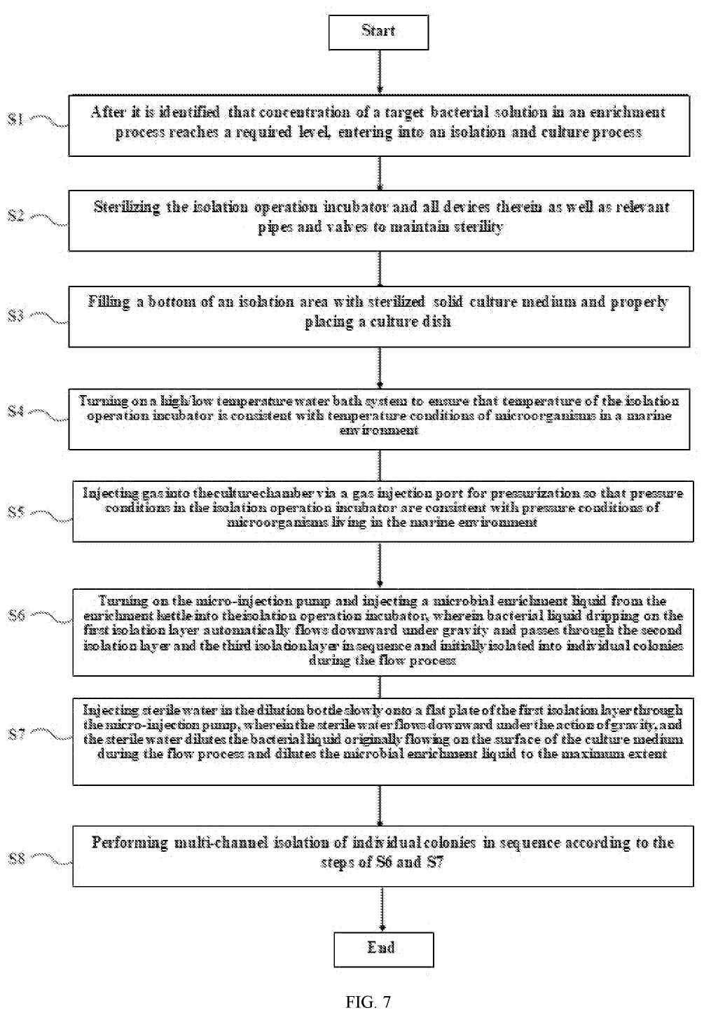

As shown in FIG. 7, the present invention further provides a method for isolating multi-type individual colonies through automatic multi-channel streaking in a high-pressure environment, by using the apparatus for isolating individual colonies through automatic multi-channel streaking in a high-pressure environment as described above. The method includes the steps of:
S1. after concentration of a target bacterial solution in an enrichment process reaches a required level, entering into an isolation and culture process;
S2. sterilizing the isolation operation incubator 2 to maintain sterility;
S3. filling a bottom of an isolation area with sterilized solid culture medium and placing a culture dish 221;
S4. turning on a water bath system to ensure that temperature of the isolation operation incubator 2 is consistent with temperature conditions of microorganisms in a marine environment;
S5. injecting gas or liquid into the isolation operation incubator 2 to pressurize the isolation operation incubator 2 so that pressure conditions in the isolation operation incubator 2 are consistent with pressure conditions of microorganisms living in the marine environment;
S6. injecting a microbial enrichment liquid from the enrichment kettle 51 into the isolation operation incubator 2 through the micro-injection pump 52, wherein bacterial liquid dripping on the first isolation layer automatically flows downward under gravity and passes through the second isolation layer and the third isolation layer in sequence and initially isolated into individual colonies during the flow process;
S7. injecting sterile water in the dilution bottle 6 onto a flat plate of the first isolation layer through the micro-injection pump 52, wherein the sterile water flows downward under the action of gravity, and the sterile water dilutes the bacterial liquid originally flowing on the surface of the culture medium during the flow process and dilutes the microbial enrichment liquid to the maximum extent; and
S8. performing multi-channel isolation of individual colonies in sequence according to the steps of S6 and S7.

Specifically, the enrichment kettle 51 and its attached pipes and valves are sterilized; then, substrates to be cultured, such as deep-sea sediments, macro-biological tissues symbiotic with microorganisms and their extracts, are sequentially fed; then, a nutrient solution necessary for incubation is fed from a liquid injection port; and a gas necessary for the incubation (or an inert gas, if not needed) is injected from a gas injection port to increase the value of the pressure in the incubation chamber to be the same as that in the actual environmental conditions of the deep sea. During the culture process, stirring is performed by means of the stirring apparatus at the top to enhance the mass transfer effect and optimize the culture process.

After it is identified that the concentration of a target microbial liquid in an enrichment process reaches a required level, an isolation and culture process is initiated. The process of isolation and culture is mainly as follows. First, the isolation operation incubator 2 and all internal devices therein as well as related pipes and valves are sterilized to maintain sterility. The culture dishes 221 are positioned on the fixing rod 211, and the connecting rods 227 are used to connect the eyelets 226 on the sides of the culture dishes 221, such that the culture dishes 221 are at a horizontal position; then, a sterilized solid culture medium is slowly filled to the culture dish 221 on the upper layer in the incubation chamber 22; and the superfluous culture medium in the first isolation layer flows into the next layer, and so on, until each culture dish 221 is filled with an equal amount of medium. The superfluous medium during the pouring flows into the liquid accumulation tank 23. After the culture medium is solidified, the connecting means is removed, such that the culture dishes 221 are tilted under the gravity, and so on, and finally the respective channels form a “Z” shape. Then, the upper cover 21 is closed; the high/low-temperature water bath system is turned on to ensure that temperature of the isolation operation incubator 2 is the same as temperature conditions of microorganisms in a marine environment. Then, gas or liquid is injected into the incubation chamber 22 via the gas injection port for pressurization to allow the pressure conditions in the isolation operation incubator 2 to be the same as the pressure conditions of microorganisms living in the marine environment. After ensuring that all system components are working normally, the micro-injection pump 52 is turned on to inject the microbial enrichment liquid from the enrichment kettle 51 into the isolation operation incubator 2, and the microbial enrichment solution drips on the surface of the culture medium on the left of the first isolation layer.

The individual colonies in the microbial enrichment liquid are isolated three times. The first isolation is performed in the first isolation layer, where the microbial liquid dripping to the first isolation layer automatically flows downward under gravity and passes through the second isolation layer and the third isolation layer in sequence, and the microbial liquid is initially isolated into individual colonies during the flow process. After a period of time (which is adjusted as appropriate), sterile water in the dilution bottle 6 is slowly injected onto a flat plate of the first isolation layer in the isolation operation incubator by the micro-injection pump 52; the sterile water gradually flows downward under gravity; and the sterile water dilutes the microbial liquid originally flowing on the surface of the culture medium during the flow process, such that the microbial enrichment liquid is diluted to the maximum extent. In order to prevent the microbial liquid on the surface of the culture medium from being completely washed off, the sterile water injected should not be excessive, and the volume of the sterile water injected should be less than the volume of the sterile water flowing out to the liquid accumulation tank 23. The superfluous microbial liquid and sterile water then enter the liquid accumulation tank 23. Each channel is operated according to the above-mentioned method, such that the automatic multi-stage isolation of individual colonies under the high-pressure environment is completed step by step under gravity. With each channel under two rounds of gravity, the probability of isolating the microbial enrichment liquid into individual colonies is improved by multiple isolation, instead of only one isolation, and depending on the type of the culture medium in the isolation and culture region, individual colonies with different conditions of nutrition can be obtained.

Based on the embodiments in the present invention, every other embodiment obtained by those of ordinary skills in the art without making creative effort shall fall within the scope of protection of the present invention.

What is claimed is:

1. An apparatus for isolating individual colonies through automatic multi-channel streaking in a high-pressure environment, comprising:
an enrichment system,
an isolation operation incubator,
a central control system,
a temperature control unit and
a pressure control unit; wherein
the enrichment system is communicated with the isolation operation incubator, and the enrichment system, the isolation operation incubator, the temperature control unit and the pressure control unit are all electrically connected to the central control system;
the enrichment system is configured to cultivate microorganisms, and the enrichment system comprises an enrichment kettle and a removable upper cover; and the enrichment system comprises a gas inlet channel or a liquid inlet channel through which gas or liquid is correspondingly injected into the enrichment kettle to pressurize the enrichment kettle;
the isolation operation incubator comprises a culture chamber wherein a plurality of culture dishes are arranged vertically or staggered horizontally and vertically, and each of the culture dishes forms a channel; and
the central control system is configured to monitor environmental data changes in the high-pressure environment, and perform monitoring, real-time collection, processing, storage and image output; the temperature control unit is configured to detect and adjust temperature changes in the enrichment system and the isolation operation incubator; and the pressure control unit is configured to detect and adjust pressure changes in the enrichment system and the isolation operation incubator, and inject gas or liquid into the isolation operation incubator for pressurization.

2. The apparatus for isolating individual colonies through automatic multi-channel streaking in a high-pressure environment according to claim 1, wherein the plurality of culture dishes is arranged in an upper layer and a lower layer, and the culture dishes in each layer are staggered;
a fixing rod perpendicular to a bottom of the culture chamber is provided in the culture chamber, and a plurality of cross bars are disposed transversely on the fixing rod;
each culture dish of the plurality culture dishes is in a shape of a rectangular parallelepiped, fixing holes are provided on either side of the culture dish, a slope is provided on the other side of the culture dish, an upper edge of the slope forms an overflow line, baffles are provided on either side of the slope, eyelets are provided below the culture dish, the eyelets are provided at an end of the culture dish close to the slope, and the plurality of culture dishes form a complete channel in a vertical direction; and
the fixing hole is provided in such a way that it extends through the cross bar, a connecting rod is provided in the eyelet, and the connecting rod is configured to connect the culture dishes arranged in the upper and lower layers.

3. The apparatus for isolating individual colonies through automatic multi-channel streaking in a high-pressure environment according to claim 2, wherein a vertical distance between each of the culture dishes is greater than or equal to a height of the culture dish itself; and when the culture dish is tilted, the culture dish forms a “Z” shape, and an inclined end of the culture dish in the upper layer is in internal contact with a non-inclined end of the culture dish in the lower layer.

4. The apparatus for isolating individual colonies through automatic multi-channel streaking in a high-pressure environment according to claim 2, wherein at least three layers of culture dishes are provided in the culture chamber, and the at least three layers of culture dishes comprise a first isolation layer, a second isolation layer and a third isolation layer; and each of the isolation layers comprises at least one of the culture dishes.

5. The apparatus for isolating individual colonies through automatic multi-channel streaking in a high-pressure environment according to claim 2, wherein the isolation operation incubator is further connected to a dilution bottle, and sterile water is injected from the dilution bottle into the culture dish.

6. The apparatus for isolating individual colonies through automatic multi-channel streaking in a high-pressure environment according to claim 1, wherein the enrichment system is communicated with the isolation operation incubator through a liquid supply pipe, and a micro-injection pump is further disposed to the liquid supply pipe; the enrichment system injects culture fluid into the isolation operation incubator through the liquid supply pipe, the micro-injection pump and spouts; and a number of the spouts is consistent with a number of culture dishes in a same plane; and
the culture chamber is further provided with a liquid accumulation tank, the liquid accumulation tank is in operative contact with the slope of the culture dish at the bottom layer; and the liquid accumulation tank is further communicated with a collection apparatus through a second pipe, and the second pipe is further provided with a valve.

7. The apparatus for isolating individual colonies through automatic multi-channel streaking in a high-pressure environment according to claim 1, wherein the enrichment kettle comprises a removable upper cover; a stirring rod is provided at the top inside the enrichment kettle; the gas inlet channel and the liquid inlet channel are equipped with valves; the enrichment kettle is further provided with a sampling port for a regulating valve; the enrichment kettle is placed in high-temperature or low-temperature water bath, or placed in an air heat exchange constant temperature room; and the enrichment system further comprises a micro-injection pump and a liquid outlet pipe.

8. A method for isolating individual colonies through automatic multi-channel streaking in a high-pressure environment using the apparatus for isolating individual colonies through automatic multi-channel streaking in a high-pressure environment according to claim 1, comprising the steps of:
S1) entering into an isolation and culture process of microorganisms after a concentration of a target bacterial solution in an enrichment process reaches a required level;
S2) sterilizing the isolation operation incubator to maintain sterility;
S3) filling a bottom of an isolation area with sterilized solid culture medium and placing a culture dish;
S4) turning on a water bath system to ensure that a temperature of the isolation operation incubator is consistent with temperature conditions of a marine environment where the microorganisms live;
S5) injecting gas or liquid into the isolation operation incubator to pressurize the isolation operation incubator so that pressure conditions in the isolation operation incubator are consistent with pressure conditions of the marine environment where the microorganisms live;
S6) injecting a microbial enrichment liquid from the enrichment kettle into the isolation operation incubator through the micro-injection pump, wherein bacterial liquid dripping on the first isolation layer automatically flows downward under gravity and passes through the second isolation layer and the third isolation layer in sequence and initially isolated into individual colonies during the flow process;
S7) injecting sterile water in the dilution bottle onto a flat plate of the first isolation layer through the micro-injection pump, wherein the sterile water flows downward under the action of gravity, and the sterile water dilutes the bacterial liquid originally flowing on the surface of the culture medium during the flow process and dilutes the microbial enrichment liquid to the maximum extent; and
S8) performing multi-channel isolation of individual colonies in sequence according to the steps of S6 and S7.

9. A method for isolating individual colonies through automatic multi-channel streaking in a high-pressure environment using the apparatus for isolating individual colonies through automatic multi-channel streaking in a high-pressure environment according to claim 2, comprising the steps of:
S1) entering into an isolation and culture process of microorganisms after a concentration of a target bacterial solution in an enrichment process reaches a required level;
S2) sterilizing the isolation operation incubator to maintain sterility;
S3) filling a bottom of an isolation area with sterilized solid culture medium and placing a culture dish;
S4) turning on a water bath system to ensure that a temperature of the isolation operation incubator is consistent with temperature conditions of a marine environment where the microorganisms live;
S5) injecting gas or liquid into the isolation operation incubator to pressurize the isolation operation incubator so that pressure conditions in the isolation operation incubator are consistent with pressure conditions of the marine environment where the microorganisms live;
S6) injecting a microbial enrichment liquid from the enrichment kettle into the isolation operation incubator through the micro-injection pump, wherein bacterial liquid dripping on the first isolation layer automatically flows downward under gravity and passes through the second isolation layer and the third isolation layer in sequence and initially isolated into individual colonies during the flow process;
S7) injecting sterile water in the dilution bottle onto a flat plate of the first isolation layer through the micro-injection pump, wherein the sterile water flows downward under the action of gravity, and the sterile water dilutes the bacterial liquid originally flowing on the surface of the culture medium during the flow process and dilutes the microbial enrichment liquid to the maximum extent; and
S8) performing multi-channel isolation of individual colonies in sequence according to the steps of S6 and S7.

10. A method for isolating individual colonies through automatic multi-channel streaking in a high-pressure environment using the apparatus for isolating individual colonies through automatic multi-channel streaking in a high-pressure environment according to claim 3, comprising the steps of:
S1) entering into an isolation and culture process of microorganisms after a concentration of a target bacterial solution in an enrichment process reaches a required level;
S2) sterilizing the isolation operation incubator to maintain sterility;
S3) filling a bottom of an isolation area with sterilized solid culture medium and placing a culture dish;
S4) turning on a water bath system to ensure that a temperature of the isolation operation incubator is consistent with temperature conditions of a marine environment where the microorganisms live;
S5) injecting gas or liquid into the isolation operation incubator to pressurize the isolation operation incubator so that pressure conditions in the isolation operation incubator are consistent with pressure conditions of the marine environment where the microorganisms live;
S6) injecting a microbial enrichment liquid from the enrichment kettle into the isolation operation incubator through the micro-injection pump, wherein bacterial liquid dripping on the first isolation layer automatically flows downward under gravity and passes through the second isolation layer and the third isolation layer in sequence and initially isolated into individual colonies during the flow process;
S7) injecting sterile water in the dilution bottle onto a flat plate of the first isolation layer through the micro-injection pump, wherein the sterile water flows downward under the action of gravity, and the sterile water dilutes the bacterial liquid originally flowing on the surface of the culture medium during the flow process and dilutes the microbial enrichment liquid to the maximum extent; and
S8) performing multi-channel isolation of individual colonies in sequence according to the steps of S6 and S7.

11. A method for isolating individual colonies through automatic multi-channel streaking in a high-pressure environment using the apparatus for isolating individual colonies through automatic multi-channel streaking in a high-pressure environment according to claim 4, comprising the steps of:
S1) entering into an isolation and culture process of microorganisms after a concentration of a target bacterial solution in an enrichment process reaches a required level;
S2) sterilizing the isolation operation incubator to maintain sterility;
S3) filling a bottom of an isolation area with sterilized solid culture medium and placing a culture dish;
S4) turning on a water bath system to ensure that a temperature of the isolation operation incubator is consistent with temperature conditions of a marine environment where the microorganisms live;
S5) injecting gas or liquid into the isolation operation incubator to pressurize the isolation operation incubator so that pressure conditions in the isolation operation incubator are consistent with pressure conditions of the marine environment where the microorganisms live;
S6) injecting a microbial enrichment liquid from the enrichment kettle into the isolation operation incubator through the micro-injection pump, wherein bacterial liquid dripping on the first isolation layer automatically flows downward under gravity and passes through the second isolation layer and the third isolation layer in sequence and initially isolated into individual colonies during the flow process;
S7) injecting sterile water in the dilution bottle onto a flat plate of the first isolation layer through the micro-injection pump, wherein the sterile water flows downward under the action of gravity, and the sterile water dilutes the bacterial liquid originally flowing on the surface of the culture medium during the flow process and dilutes the microbial enrichment liquid to the maximum extent; and
S8) performing multi-channel isolation of individual colonies in sequence according to the steps of S6 and S7.

12. A method for isolating individual colonies through automatic multi-channel streaking in a high-pressure environment using the apparatus for isolating individual colonies through automatic multi-channel streaking in a high-pressure environment according to claim 5, comprising the steps of:
S1) entering into an isolation and culture process of microorganisms after a concentration of a target bacterial solution in an enrichment process reaches a required level;
S2) sterilizing the isolation operation incubator to maintain sterility;
S3) filling a bottom of an isolation area with sterilized solid culture medium and placing a culture dish;
S4) turning on a water bath system to ensure that a temperature of the isolation operation incubator is consistent with temperature conditions of a marine environment where the microorganisms live;
S5) injecting gas or liquid into the isolation operation incubator to pressurize the isolation operation incubator so that pressure conditions in the isolation operation incubator are consistent with pressure conditions of the marine environment where the microorganisms live;
S6) injecting a microbial enrichment liquid from the enrichment kettle into the isolation operation incubator through the micro-injection pump, wherein bacterial liquid dripping on the first isolation layer automatically flows downward under gravity and passes through the second isolation layer and the third isolation layer in sequence and initially isolated into individual colonies during the flow process;
S7) injecting sterile water in the dilution bottle onto a flat plate of the first isolation layer through the micro-injection pump, wherein the sterile water flows downward under the action of gravity, and the sterile water dilutes the bacterial liquid originally flowing on the surface of the culture medium during the flow process and dilutes the microbial enrichment liquid to the maximum extent; and
S8) performing multi-channel isolation of individual colonies in sequence according to the steps of S6 and S7.

13. A method for isolating individual colonies through automatic multi-channel streaking in a high-pressure environment using the apparatus for isolating individual colonies through automatic multi-channel streaking in a high-pressure environment according to claim 6, comprising the steps of:
S1) entering into an isolation and culture process of microorganisms after a concentration of a target bacterial solution in an enrichment process reaches a required level;
S2) sterilizing the isolation operation incubator to maintain sterility;
S3) filling a bottom of an isolation area with sterilized solid culture medium and placing a culture dish;
S4) turning on a water bath system to ensure that a temperature of the isolation operation incubator is consistent with temperature conditions of a marine environment where the microorganisms live;
S5) injecting gas or liquid into the isolation operation incubator to pressurize the isolation operation incubator so that pressure conditions in the isolation operation incubator are consistent with pressure conditions of the marine environment where the microorganisms live;
S6) injecting a microbial enrichment liquid from the enrichment kettle into the isolation operation incubator through the micro-injection pump, wherein bacterial liquid dripping on the first isolation layer automatically flows downward under gravity and passes through the second isolation layer and the third isolation layer in sequence and initially isolated into individual colonies during the flow process;
S7) injecting sterile water in the dilution bottle onto a flat plate of the first isolation layer through the micro-injection pump, wherein the sterile water flows downward under the action of gravity, and the sterile water dilutes the bacterial liquid originally flowing on the surface of the culture medium during the flow process and dilutes the microbial enrichment liquid to the maximum extent; and
S8) performing multi-channel isolation of individual colonies in sequence according to the steps of S6 and S7.

14. A method for isolating individual colonies through automatic multi-channel streaking in a high-pressure environment using the apparatus for isolating individual colonies through automatic multi-channel streaking in a high-pressure environment according to claim 7, comprising the steps of:
S1) entering into an isolation and culture process of microorganisms after a concentration of a target bacterial solution in an enrichment process reaches a required level;
S2) sterilizing the isolation operation incubator to maintain sterility;
S3) filling a bottom of an isolation area with sterilized solid culture medium and placing a culture dish;
S4) turning on a water bath system to ensure that a temperature of the isolation operation incubator is consistent with temperature conditions of a marine environment where the microorganisms live;
S5) injecting gas or liquid into the isolation operation incubator to pressurize the isolation operation incubator so that pressure conditions in the isolation operation incubator are consistent with pressure conditions of the marine environment where the microorganisms live;
S6) injecting a microbial enrichment liquid from the enrichment kettle into the isolation operation incubator through the micro-injection pump, wherein bacterial liquid dripping on the first isolation layer automatically flows downward under gravity and passes through the second isolation layer and the third isolation layer in sequence and initially isolated into individual colonies during the flow process;
S7) injecting sterile water in the dilution bottle onto a flat plate of the first isolation layer through the micro-injection pump, wherein the sterile water flows downward under the action of gravity, and the sterile water dilutes the bacterial liquid originally flowing on the surface of the culture medium during the flow process and dilutes the microbial enrichment liquid to the maximum extent; and
S8) performing multi-channel isolation of individual colonies in sequence according to the steps of S6 and S7.
